(19) United States
(12) Patent Application Publication
(10) Pub. No.: US 2008/0041393 A1
(43) Pub. Date: Feb. 21, 2008

(54) Nasal Cannula For The Delivery of Humidified Oxygen

(76) Inventor: Ronny Bracken, Monroe, GA (US)

Correspondence Address: BRIAN M. BURN, ESQ.;C. R. BARD MEDICAL DIVISION, P.O. BOX 52050, c/o PORTFOLIOIP, MINNEAPOLIS, MN, 55402, US

(21) Appl. No.: 11/828,663

(22) Filed: Jul. 26, 2007

Related U.S. Application Data
(60) Provisional application No. 60/821,879, filed on Aug. 9, 2006.

(51) Int. Cl. A61M 15/08

(52) U.S. Cl. 128/207.18

(57) ABSTRACT

A nasal cannula assembly for the delivery of a gas to a patient The nasal cannula assembly includes a nasal cannula having a pair of apertured nostril outlet prongs, a first gas inlet and a second gas inlet, a first gas inlet tube formed of a flexible polymer having a first end and a second end, the first end in fluid communication with the first gas inlet of the of nasal cannula, a second gas inlet tube formed of a flexible polymer having a first end and a second end, the first end in fluid communication with the second gas inlet of the of nasal cannula and a one-way check valve positioned within a gas flowpath of either the first gas inlet tube or the second gas inlet tube. A method of delivering a gas to a patient is also provided.

The present application claims priority to U.S. Provisional Application No. 60/821,879, filed Aug. 9, 2006, the disclosure of which is incorporated herein by reference in its entirety

The present disclosure relates to an apparatus and method for respiratory tract therapy. More particularly, this disclosure relates to a nasal cannula for the delivery of humidified gas to the respiratory tract of a patient.

Oxygen therapy is a key treatment in respiratory care. Such therapy serves to increase oxygen saturation in tissues where the saturation levels are too low due to illness or injury Some of the conditions in which oxygen therapy is used include hypoxemia, severe respiratory distress (e g., acute asthma or pneumonia), severe trauma, acute myocardial infarction and short-term therapy, such as post-anesthesia recovery. Hyperbaric oxygen therapy is used in cases of gas gangrene, decompression sickness, air embolism, smoke inhalation, carbon monoxide poisoning and cerebral hypoxic events.

In the delivery of oxygen and oxygen-enriched air, it is recognized that significant discomfort is often experienced by the patient, especially when the air is delivered over an extended period of time. Moreover, it is generally known that it is far more beneficial for the patient to receive such gases under conditions of somewhat elevated heat and humidity, rather than to supply the patient with a cool dry gas. It has also been recognized that the delivery of air having relatively low absolute humidity can result in respiratory irritation. It has been found, for example, that when the inhaled gas is both heated and humidified, the patient is more receptive to the gas, with other potential respiratory diseases minimized.

Nasal cannula assemblies have found widespread use in providing oxygen and other gases to a patient. Such assemblies have largely replaced oxygen masks and provide much greater comfort than nasal catheters. The use of such devices has proved sufficiently beneficial so that they are widely used not only by respiratory patients, but also for a wide variety of patients who require less energy to breathe with the added oxygen supplied by such assemblies. The most commonly used arrangement includes a dual prong nose piece which is centered in a loop of vinyl tubing. The nose piece openings are inserted in the nose with the tubing tucked behind the ears. A slide adjustment may be used to draw it tight beneath the chin.

Conventional nasal cannula do not allow for true positive pressure ventilation, since both nares are open to the atmosphere As conventional nasal cannula systems do not provide a positive seal between the nasal outlet prongs and the nostrils, nasal ventilation systems often include a mask that fits over the nose that is intended to provide a space of oxygen-enriched air for inhalation into the lungs for respiration. These systems frequently suffer from air leaking from around the mask, creating an inability to assure ventilation in many patients. Additionally, these systems are often very position dependent, whereby if the mask is moved slightly with respect to the facial contour or with respect to the nose, air leakage occurs With such systems, the mask can become uncomfortable when not in position, thus requiring the patient to remain rather still in order to alleviate the discomfort and to maintain oxygen inspiration

In a system proposed for use in sleep apnea patients, an integrally molded ventilation interface was proposed to provide a positive airway pressure and address the sealing issue The interface proposed includes a hollow bellows-like structure and two nasal prongs extending from a top surface of the bellows. A pair of headgear strap flanges may also be molded integrally with the ventilation interface. The nasal prongs proposed are said to provide a first sealing interface between an outer surface of the nasal prongs and an inner surface of the patient's nares. The bellows is said to provide a second sealing interface between a top surface of the bellows-like structure and a bottom surface of a patient's nose. The headgear strap flanges provide a third sealing interface between the ventilation interface and a mustache region of the patient's face as well as a bottom surface of the patient's nose.

As may be appreciated, when delivering heated, humidified oxygen, the surface area traversed by the oxygen, if excessive, will cause a high degree of heat loss from the oxygen, resulting in excessive moisture dropout. As such, certain solutions proposed for achieving positive airway pressure and sealing are ineffective in the delivery of heated, humidified oxygen to the respiratory tract of a patient.

As such, there remains a need for an improved nasal cannula and apparatus for respiratory tract therapy that overcomes the problems associated with current designs.

In one aspect, provided is a nasal cannula assembly for the delivery of a gas to a patient. The nasal cannula assembly includes a nasal cannula having a pair of apertured nostril outlet prongs, a first gas inlet and a second gas inlet, a first gas inlet tube formed of a flexible polymer having a first end and a second end, the first end in fluid communication with the first gas inlet of the of nasal cannula, a second gas inlet tube formed of a flexible polymer having a first end and a second end, the first end in fluid communication with the second gas inlet of the of nasal cannula and a one-way check valve positioned within a gas flowpath of either the first gas inlet tube or the second gas inlet tube.

In another aspect, provided is a method of delivering a gas to a patient. The method includes the steps of placing a nasal cannula assembly in communication with an airway of a patient, the nasal cannula assembly comprising a nasal cannula having a pair of apertured nostril outlet prongs, a first gas inlet and a second gas inlet, a first gas inlet tube formed of a flexible polymer having a first end and a second end, the first end in fluid communication with the first gas inlet of the of nasal cannula, a second gas inlet tube formed of a flexible polymer having a first end and a second end, the first end in fluid communication with the second gas inlet of the of nasal cannula, and a one-way check valve positioned within a gas flowpath of either the first gas inlet tube or the second gas inlet tube, and delivering a gas to the nasal cannula assembly

In one embodiment, the first and second gas inlet tubes are formed of a flexible polymer chosen from polyvinyl chloride, polyurethane, polyethylene, polypropylene, polyester, copolymers, terpolymers and blends thereof.

In another embodiment, the one-way check valve includes an elongated valve body having a central longitudinal axis and an outer wall wherein the outer wall is substantially parallel to the axis and defines an axial and circumferential extent of the second end of the valve, a pair of valve lips positioned within the axial and circumferential extent of the second end defined by the outer wall portion of the valve body and oriented in diverging relationship to each other toward the first end of the valve, the valve body and lips being formed of an elastomeric material and wherein the lips define an elongated normally closed outlet opening of the valve at the second end, and a pivoting connection between the outer wall and the lips including a pair of connecting walls located on either side of the outlet opening and extending radially inwardly from the outer wall to intersect the lips along a smoothly curved edge

In yet another embodiment, the nasal cannula further includes a flexible bladder covering to seal both nares of the patient. The flexible bladder includes a nasal cannula bladder body portion and a pair of nostril outlet prong bladder portions. The flexible bladder may be inflated to seal the nares of a patient and may be made of a soft rubber-like material.

These and other features will be apparent from the description taken with reference to accompanying drawings

### BRIEF DESCRIPTION OF THE DRAWINGS

The invention is further explained in the description that follows with reference to the drawings illustrating, by way of non-limiting examples, various embodiments of the disclosure wherein:

FIG. 1 is a frontal elevational view of one embodiment of a nasal cannula assembly having a one-way check valve and a flexible bladder covering;

FIG. 2 is an exploded perspective view of a one-way check valve for use in the nasal cannula assemblies disclosed herein;

FIG. 3 is a plan view of the outlet end of a regulator portion of the one-way check valve of FIG. 2; and

FIG. 4 is a is a sectional view showing the assembled one-way check valve of FIG. 2.

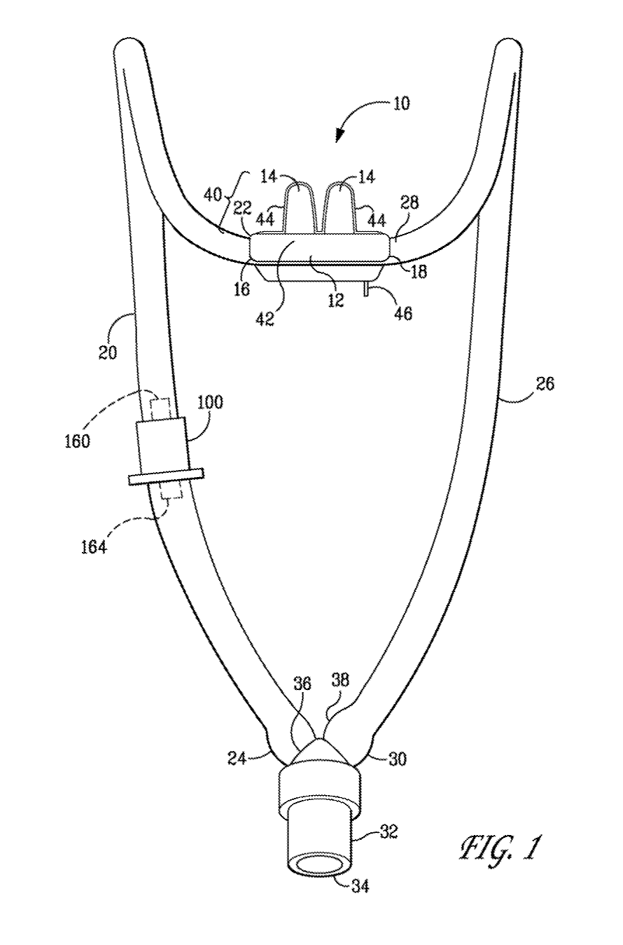

Various aspects will now be described with reference to specific embodiments selected for purposes of illustration. It will be appreciated that the spirit and scope of the nasal cannula assembly disclosed herein is not limited to the selected embodiments. Moreover, it is to be noted that the figures provided herein are not drawn to any particular proportion or scale, and that many variations can be made to the illustrated embodiments. Reference is now made to FIGS. 1-4, wherein like numerals are used to designate like parts throughout.

Referring now to FIG. 1, an exemplary embodiment of a nasal cannula assembly 10 is depicted that includes a nasal cannula 12 having a pair of apertured nostril outlet prongs 14, a first gas inlet 16 and a second gas inlet 18. Nasal cannula assembly 10 also includes a first gas inlet tube 20 having a first end 22 and a second end 24, first end 22 being in fluid communication with the first gas inlet 16 of nasal cannula 12. Likewise, nasal cannula assembly 10 also includes a second gas inlet tube 26 having a first end 28 and a second end 30, first end 28 being in fluid communication with second gas inlet 18 of nasal cannula 12.

The second end 24 of first gas inlet tube 20 and the second end 30 of second gas inlet tube 26 are connected to outlets 36 and 38, respectively, of connector 32. During use, a gas supply conduit (not shown) is connected to connector inlet 34 and to a source of pressurized gas, such as oxygen, air or mixtures thereof

First gas inlet tube 20 and second gas inlet tube 26 may be formed from a variety of materials and by a variety of processes. First gas inlet tube 20 and second gas inlet tube 26 may be formed from a polymeric material such as polyvinyl chloride, polyurethane, polyethylene, polypropylene, polyester and blends thereof. First gas inlet tube 20 and second gas inlet tube 26 may also be formed from a polyether urethane, such as Pellethane® 2363-80AE. Pellethane® is available from The Dow Chemical Company of Midland, Mich. First gas inlet tube 20 and second gas inlet tube 26 can be clear, or substantially clear, to enable a user to ascertain that no condensation occurs within either tube. First gas inlet tube 20 and second gas inlet tube 26 can be extruded in lengths having a substantially constant cross-sectional shape

As may be appreciated by those skilled in the art, conventional nasal cannula assemblies do not allow for true positive pressure ventilation, since both nares are open to the atmosphere. To address this issue in part, nasal cannula assembly 10 is provided with a one-way check valve 100 positioned within a gas flowpath of either first gas inlet tube 20, as shown, or within second gas inlet tube 26, or within both inlet tubes

A wide variety of one-way check valves have utility in the nasal cannula assemblies disclosed herein One of the requirements for a one-way check valve is that the valve should permit fluid flow in one direction but stop fluid flow in the opposite direction. One such one-way check valve contemplated for use herein is a duckbill valve

A typical duckbill valve includes a resilient flow regulator member mounted in a fluid flow path and has as its primary operative components a pair of lips arranged in a converging relationship from an inlet end at the base of the lips to an outlet end At the outlet end of the regulator, the lips are located adjacent to each other so as to define a slit therebetween The duckbill regulator is often mounted within a housing in a sealed relationship so that flow through the housing must pass through the regulator as well. In a forward direction, flow passes into the regulator through the inlet end, moving toward the slit formed at the outlet end. The flow pressure against the resilient lips opens the slit, allowing the flow to pass out of the regulator. When flow enters the duckbill regulator from a reverse direction, the flow contacts the regulator lips at its outlet end, with the flow pressure against the resilient lips holding the slit in a closed position, thereby preventing flow through the valve.

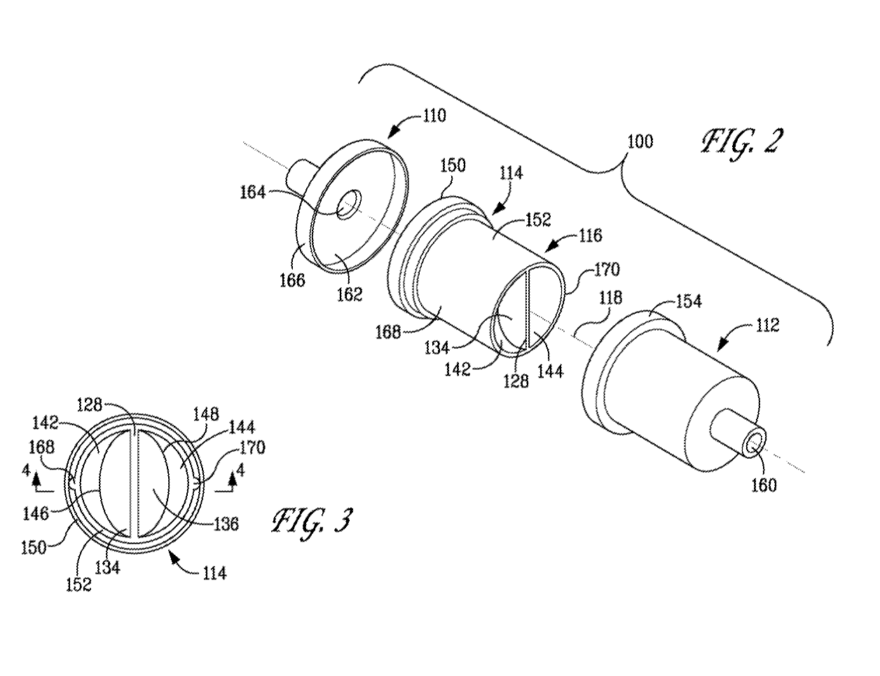

Reference is now made to FIGS. 2-4, wherein the one-way check valve 100 of FIG. 1 and its operation will be described in more detail One-way check valve 100 includes a housing inlet portion 110, a housing outlet portion 112 and a regulator portion 114 which may be located within the outlet portion 112, as shown. The portions 110 and 112 may be molded from a transparent acrylic plastic material, although other materials can also be used. Flow regulator portion 114 may be molded as a single piece from a material having elastic properties, such as an elastomeric material.

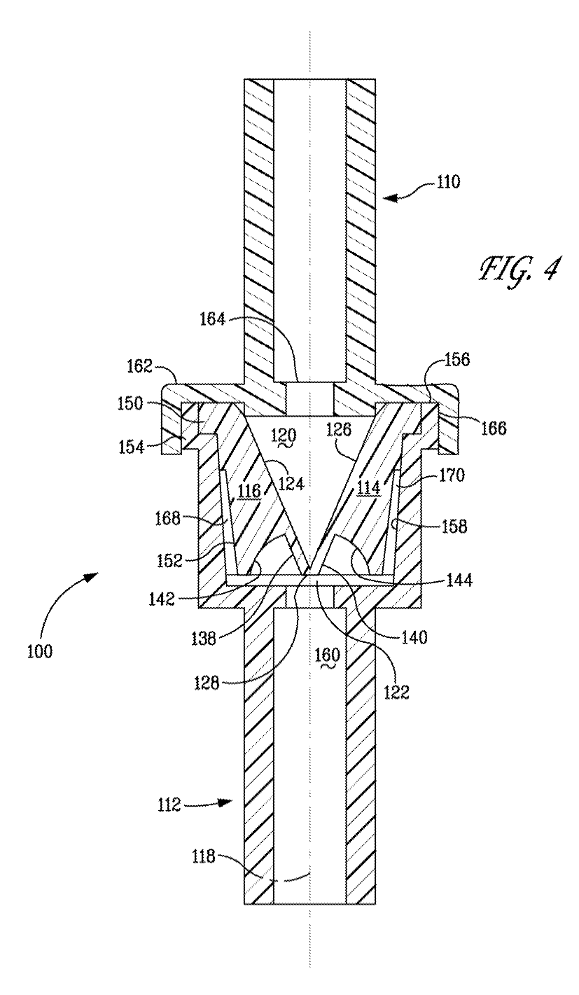

Flow regulator 114 includes a main body 116 which may be substantially cylindrical and which defines a central longitudinal axis 118 of the one-way check valve 100. The regulator portion 114 is formed as a hollow member to define a flow path from an inlet end 120 to an outlet end 122 of the regulator portion 114. The regulator portion 114 further includes a pair of substantially planar inner walls 124 and 126, as may be seen in FIG. 4, which are arranged in converging relationship and extend through the interior of the main body 116 from the inlet end 120 to the outlet end 122. At the outlet end 122 the inner walls 124 and 126 are disposed adjacent to each other to define a normally closed elongated slit 128 therebetween which is bisected by the axis 118. The inner walls are interconnected along the length of the main body 116 by a pair of generally curved side wall portions 130 and 132 extending along the main body 114

A pair of generally planar outer walls 134 and 136 are disposed approximately parallel to the converging inner walls 124 and 126 and extend in diverging relationship toward the inlet end 120 of the regulator portion 114 from a point adjacent to the slit 128 at the outlet end 122. The inner and outer walls together define a pair of lips 138 and 140 which converge from the inlet to the outlet end of the valve regulator portion 114.

A pair of connecting walls 142 and 144 having concave surfaces are located on either side of the slit 128 and extend from the main body 116 to intersect the lips 138 and 140 at a point intermediate the inlet and outlet ends 120 and 122 of the regulator portion 114 The intersection of the connecting walls 142 and 144 with the lips 138 and 140 forms a pivot portion for each of the lips 138 and 140 to pivot away from each other so as to allow a fluid flow through the regulator portion 114 in a first direction from the inlet to the outlet end In addition, the connecting walls 142 and 144 and lips 138 and 140 define a pair of cavities which extend into the main body from the outlet end 122 on either side of the slit 128.

As may be seen in FIGS. 3 and 4, the connecting walls 142 and 144 are each defined by a locus of points which are generally equidistant from a predetermined center of curvature located along the center axis 18 whereby the intersection of the connecting walls 142 and 144 with the lips 138 and 140 define a pair of generally semi-circular lines 146 and 148, as viewed in a direction perpendicular to the planes containing the lips 138 and 140, respectively. Connecting walls 142 and 144 are formed with a generally semi-circular shape and are biased into a closed position and supported for pivotal movement along the semi-circular lines 146 and 148.

Referring to FIG. 4, the regulator portion 114 may be provided with a flange 150 extending radially outwardly beyond an outer wall 152 defining an outer circumferential extent of the main body 116 The outlet portion 112 of the housing is provided with a collar 154 supported by a shelf 156 extending around the periphery of the outlet portion 112. The shelf 156 and collar 154 define an annular seat for receiving the flange 150 so that the regulator portion 114 may be positioned within the outlet portion 112. When the flange is in place on the valve seat of the outlet portion 112, the outer wall 152 of the main body 116, which extends substantially parallel to the axis 118 and which defines an axial extent of the main body 116, is held in spaced relation to the interior surface 158 of the outlet portion 112 and the outlet end 122 is held adjacent to an outlet port 160

The inlet portion 110 includes a substantially circular cover plate 162 through which an inlet port 164 extends for allowing passage of fluid flow to the regulator portion 114. A circular sealing ring 166 extends perpendicularly from the cover plate 162. As may be seen in FIG. 4, the cover plate 162 engages a surface of the flange 150 whereby the flange is pressed onto the shelf surface 156 and the sealing ring 166 engages an outer surface of the collar 154 to thereby form a seal so that fluid flow is forced to flow through the inlet port 164, through the inlet end 120 of the regulator 114 and through the slit 128 to the outlet port 160.

In order to provide a biasing force whereby the lips 138 and 140 are forced together into a closed position, the main body 116 is provided with a pair of ribs 168 and 170 which protrude radially from the outer wall 152 of the main body 116. The ribs 168 and 170 extend parallel to the axis 118 in a plane containing the axis 118 and oriented perpendicular to the slit 128. The diameter of the outer wall 152 and the dimensions of the ribs 168 and 170 are selected such that the ribs 168 and 170 will engage the interior surface 158 of the outlet portion 112 in an interference fit whereby the main body is biased inwardly at the location of the ribs 168 and 170. As a result of the biasing force applied to the main body 116, the connecting walls 142 and 144 are caused to move inwardly toward each other whereby a greater spring force is produced along the semi-circular lines 146 and 148 to positively bias the lips 138 and 140 together without restricting their pivotal movement.

It should be apparent that by forming the ribs 168 and 170 such that they extend an appropriate radial distance from the outer wall 152 of the main body 116, the amount of biasing force applied to the lips 138 and 140 and therefore the amount of forward flow pressure required to initiate flow through the lips may be precisely controlled. In addition, the radius of curvature of the concave surfaces forming the connecting walls 142 and 144 may also be varied to alter the amount of biasing force applied to the lips 138 and 140 as the outer wall 152 of the main body 116 is biased inwardly.

Lip portions 138, 140 of one-way check valve 100 provide a biasing portion formed adjacent to the semi-circular lines 146 and 148, which are located relatively close to the outlet slit 128 to provide a positive biasing force to the lips 138 and 140, while also including the flexible lip portions 138 and 140 which allow relatively unrestricted flow through the outlet 122 of the regulator 114. In addition, by providing the cavities on either side of the slit 128, back pressure resulting from a reverse flow condition will act on the outer surfaces 134 and 136 of the lips 138 and 140 to further force the lips 138 and 140 together and thereby prevent reverse flow through the valve.

The relative dimensions of the interior surface 158 of the housing outlet portion 112 and the main body 116 may be selected so that a predetermined inward biasing force is produced on the lips 138 and 140 whereby a predetermined forward flow pressure is required in order to initiate flow in the first direction from the inlet 120 to the outlet 122. In such a construction, once the forward flow has been initiated, the flow will continue in a relatively unrestricted manner until the pressure drops below the predetermined level at which time the valve lips will shut, even in the absence of reverse fluid flow. Thus, one-way check valve 100 is not dependent upon reverse flow pressure to bias the lips 138 and 140 together to close the slit 128 for preventing reverse flow.

As indicated above, a characteristic of one-way check valve 100 is the ability to control the forward flow pressure at which the valve will open. In order to assure that adequate positive pressure is achieved by nasal cannula assembly 10, one-way check valve 100 may be pre-loaded to urge it shut. The pre-load can range from quite small or can be larger to establish a significant, positive pressure within nasal cannula assembly 10. One-way check valve 100 may be pre-loaded to start functioning at about 10 psi to about 50 psi to assure positive pressure during the supply of air, oxygen or mixtures thereof.

Suitable pre-loaded one-way check valves may be obtained from Vernay Laboratories, Inc. of Yellow Springs, Ohio. Other sources of one-way duckbill valves include Minivalve International of Oldenzaal, Netherlands, Da/Pro Rubber, Inc. of Valencia, Calif. and others. As indicated above, other one-way check valve designs have utility in the nasal cannula assembly disclosed herein.

Referring again to FIG. 1, to further address the issue that conventional nasal cannula assemblies do not allow for true positive pressure ventilation, since both nares are open to the atmosphere, nasal cannula 12 of nasal cannula assembly 10 may also include a flexible bladder covering 40 that includes a nasal cannula bladder body portion 42 and a pair of nostril outlet prong bladder portions 44 In use, flexible bladder covering 40 is inflated with air, sterile water or the like at inflation member 46 to seal both nares of a patient undergoing respiratory tract treatment. Flexible bladder covering 40 may be made of a soft rubber-like material, for patient comfort.

The nasal cannula assembly 10 described herein may be employed with an apparatus that provides a source for heated, humidified air, oxygen or blends thereof (not shown). Such an apparatus may be adapted for use in a variety of settings and for transport between locations. The apparatus may be used in the home by a patient and at the patients bedside, if desired. Such an apparatus can also be used in hospitals, clinics, and other settings, as well The nasal cannula assembly 10 may be designed so that it can be used by a particular patient and then discarded after one or any number of uses

The nasal cannula assembly 10 provides a passageway for the flow of humidified gas to the patient's respiratory tract. In operation, gas (air, oxygen, or some combination) is supplied at about 50 psi maximum pressure. The gas flow can be regulated by a user-supplied restricting valve at the source of the gas so that it can be controlled between flows of about 5 to 50 l/min, or between about 5 to 40 l/min. A nasal cannula assembly 10 can be attached to a gas delivery tube that is attached at the front of the aforementioned apparatus via a manifold (not shown) that interfaces with a gas supply port. The apparatus can be designed to operate on standard 115VAC, 60 Hz. The apparatus can also employ a microprocessor to control heating, humidification, gas flow, gas pressure, etc., as those skilled in the art will readily understand.

The contemplated apparatus for the supply of heated, humidified air, oxygen or blends thereof, is adapted to operate within predetermined parameters. In one exemplary embodiment, such an apparatus can operate in a controlled air output temperature range of from about 35° C. to about 43° C.; an operating flow range of about 5 to about 40 l/min.; a gas pressure not to exceed about 60 psi; and a gas composition of dry air and/or oxygen, from about 21% O₂ to about 100% O₂. Gas humidification may exceed about 95% relative humidity.

The nasal cannula assemblies disclosed herein can yield significant benefits when used for the treatment of the respiratory tract or for respiratory tract therapy. As indicated, the nasal cannula assemblies disclosed herein can be adapted for the introduction of heated and humidified air to the respiratory tract of a human patient. Home use, hospital and clinical use are contemplated.

The introduction of heated and humidified air by using the nasal cannula assemblies disclosed herein can provide several unique advantages as compared to conventional nasal cannulas in connection with the treatment of respiratory tract conditions. The use of the nasal cannula assemblies disclosed herein can ensure that saturated air is delivered to the nose at body temperature or higher without heat loss or condensation, and a high flow rate of heated and humidified air ensures that almost all of the air breathed by a patient is heated and humidified with little or no entrained room air. These benefits can be accomplished by delivering air through a nasal cannula of the type disclosed herein so that the patient can continue normal activities with minimal interference.

The nasal cannula assemblies disclosed herein can provide relief to people who suffer from e.g., asthma Conventionally, asthma sufferers are recommended to keep humidity low because dust mites are more common in moist environments. Accordingly, the nasal cannula assemblies disclosed herein provide the benefits of warm humid air in the entire respiratory tract, without the problems associated with high ambient humidity. According to various embodiments, the nasal cannula assembly can provide relief to people suffering from sleep apnea.

A supply of room air saturated with water vapor at about 40° C. directly to the airway via a nasal cannula of the type disclosed herein, thereby avoiding problems of condensation and cooling associated with conventional delivery of humidified air, reduces nasal irritation by eliminating drying and cooling of the nasal mucosa and pharynx, and is therefore therapeutic for asthma and rhinitis.

All patents, test procedures, and other documents cited herein, including priority documents, are fully incorporated by reference to the extent such disclosure is not inconsistent with this invention and for all jurisdictions in which such incorporation is permitted.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the invention, including all features which would be treated as equivalents thereof by those skilled in the art to which the invention pertains.

What is claimed is:

1. A nasal cannula assembly for the delivery of a gas to a patient, comprising:
(a) a nasal cannula having a pair of apertured nostril outlet prongs, a first gas inlet and a second gas inlet;
(b) a first gas inlet tube formed of a flexible polymer having a first end and a second end, said first end in fluid communication with said first gas inlet of said of nasal cannula;
(c) a second gas inlet tube formed of a flexible polymer having a first end and a second end, said first end in fluid communication with said second gas inlet of said of nasal cannula; and
(d) a one-way check valve positioned within a gas flowpath of, at least one of said first gas inlet tube and said second gas inlet tube.

2. The nasal cannula assembly of claim 1, wherein said flexible polymer is chosen from polyvinyl chloride, polyurethane, polyethylene, polypropylene, polyester, copolymers, terpolymers and blends thereof.

3. The nasal cannula assembly of claim 1, wherein said one-way check valve comprises
(i) an elongated valve body having a central longitudinal axis and an outer wall wherein said outer wall is substantially parallel to said axis and defines an axial and circumferential extent of said second end of said valve;
(ii) a pair of valve lips positioned within the axial and circumferential extent of said second end defined by said outer wall portion of said valve body and oriented in diverging relationship to each other toward said first end of said valve; said valve body and lips being formed of an elastomeric material and wherein said lips define an elongated normally closed outlet opening of said valve at said second end; and
(iii) a pivoting connection between said outer wall and said lips including a pair of connecting walls located on either side of said outlet opening and extending radially inwardly from said outer wall to intersect said lips along a smoothly curved edge.

4. The nasal cannula assembly of claim 3, wherein the intersection of said connecting walls with said lips of said one-way check valve forms a pivot point for each of said lips to pivot away from said axis to allow flow in the flowpath.

5. The nasal cannula assembly of claim 3, wherein said elongated outlet opening intersects said axis of said one-way check valve, and said connecting walls intersect said lips along a locus of point substantially equidistant from a predetermined center of curvature.

6. The nasal cannula assembly of claim 5, wherein said connecting walls of said one-way check valve are formed with concave surfaces and said concave surfaces are defined by a locus of points substantially equidistant from said predetermined center of curvature.

7. The nasal cannula assembly of claim 3, wherein said outer wall of said one-way check valve includes enlarged portions located in a plane containing the axis and oriented perpendicular to said outlet opening, wherein said enlarged portions extend radially from said valve body

8. The nasal cannula assembly of claim 7, wherein said enlarged portions of said one-way check valve comprise a pair of diametrically opposed ribs

9. The nasal cannula assembly of claim 8, wherein said nasal cannula further includes a flexible bladder covering to seal both nares of the patient

10. The nasal cannula assembly of claim 1, wherein said nasal cannula further includes a flexible bladder covering to seal both nares of the patient

11. The nasal cannula assembly of claim 10, wherein said flexible bladder includes a nasal cannula bladder body portion and a pair of nostril outlet prong bladder portions

12. The nasal cannula assembly of claim 10, wherein said flexible bladder is inflated to seal both nares of a patient

13. The nasal cannula assembly of claim 10, wherein said flexible bladder is made of a soft rubber-like material.

14. The nasal cannula assembly of claim 1, further comprising a connector having a first outlet and a second outlet, wherein said second end of said first gas inlet tube is connected to said first outlet and said second end of said second gas inlet tube is connected to said second outlet.

15. A method of delivering a gas to a patient, the method comprising the steps of:
(a) placing a nasal cannula assembly in communication with an airway of a patient; the nasal cannula assembly comprising a nasal cannula having a pair of apertured nostril outlet prongs, a first gas inlet and a second gas inlet; a first gas inlet tube formed of a flexible polymer having a first end and a second end, the first end in fluid communication with the first gas inlet of the of nasal cannula; a second gas inlet tube formed of a flexible polymer having a first end and a second end, the first end in fluid communication with the second gas inlet of the of nasal cannula; and a one-way check valve positioned within a gas flowpath of, at least one of the first gas inlet tube and the second gas inlet tube; and
(b) delivering a gas to the nasal cannula assembly

16. The method of claim 15, wherein the flexible polymer is chosen from polyvinyl chloride, polyurethane, polyethylene, polypropylene, polyester, copolymers, terpolymers and blends thereof.

17. The method of claim 15, wherein the one-way check valve comprises
(i) an elongated valve body having a central longitudinal axis and an outer wall wherein the outer wall is substantially parallel to the axis and defines an axial and circumferential extent of the second end of the valve;
(ii) a pair of valve lips positioned within the axial and circumferential extent of the second end defined by the outer wall portion of the valve body and oriented in diverging relationship to each other toward the first end of the valve; the valve body and lips being formed of an elastomeric material and wherein the lips define an elongated normally closed outlet opening of the valve at the second end; and
(iii) a pivoting connection between the outer wall and the lips including a pair of connecting walls located on either side of the outlet opening and extending radially inwardly from the outer wall to intersect the lips along a smoothly curved edge.

18. The method of claim 17, wherein the intersection of the connecting walls with the lips of the one-way check valve forms a pivot point for each of the lips to pivot away from the axis to allow flow in the flowpath.

19. The method of claim 17, wherein the elongated outlet opening intersects the axis of the one-way check valve, and the connecting walls intersect the lips along a locus of point substantially equidistant from a predetermined center of curvature.

20. The method of claim 19, wherein the connecting walls of the one-way check valve are formed with concave surfaces and the concave surfaces are defined by a locus of points substantially equidistant from the predetermined center of curvature.
